(19) United States
(12) Plant Patent Application Publication
(10) Pub. No.: US 2002/0032920 P1
(43) Pub. Date: Mar. 14, 2002

(54) Apple tree named 'Swedes Fuji'

(76) Inventors: Dennis K. Pickering, Oroville, WA (US); Sterling G. Pickering, Tonasket, WA (US)

Correspondence Address: Dennis K. Pickering, 32292 Hwy 97, Rt 2, Box 884, Oroville, WA, 98844, US

(21) Appl. No.: 09/905,663

(22) Filed: Jul. 16, 2001

Related U.S. Application Data
(60) Provisional application No. 60/221,883, filed on Jul. 31, 2000.

(51) Int. Cl. A01H 5/00

(52) U.S. Cl. PLT/168

(57) ABSTRACT

A new and distinct late season variety of apple tree, named ‘Swedes Fuji’ originating from a mutation of the standard Fuji. This new cultivar displays a brilliant red blush uniform coloration which extends over the entire surface of the apple. The cultivar is generally similar to the parent with the exception of: 1) fruit color and 2) it is easier to harvest because it has a more uniform rate of coloration throughout the entire tree when the fruit is maturing in late fall.

### BACKGROUND OF INVENTION

The Okanogan Valley in Washington state, where the ‘Swedes Fuji’ apple trees are now growing, is an agricultural region geographically structured into primarily small family farms. Our own family has been commercially producing apples of many different varieties for five generations now, and the present generation of our Pickering family continues this tradition. We have been experimenting and developing new varieties of apple trees for 80 years and have upgraded our orchards whenever improvements were found.

Unfortunately, no written records were kept for most of those years. Also, some of the earlier helpful apple growers in the family, as well as supportive non-family apple growing friends are now deceased. My point in stating this is that our current orchard makeup, which is the best that we have developed in many years, is the result of not only the current inventors, but is also at least partially because of the efforts of a few of the deceased—even though no records exist.

We were among the very first people to introduce Fujis into valley during the 1980s at which time we grafted and planted in as many different types of Fujis as was then available. We have been experimenting and grafting every since on thousands of trees comprised of seven different varieties of Fujis now growing. During the course of those earlier years (it was either 1990 or 1991) we noticed at harvest time that the fruit from one or two trees was quite different than all the others and we have concentrated our efforts on scion taken from that source. The progeny has been stable for three grafting generations now. This new cultivar is generally similar to standard Fujis in all respects except for color. It has a very intense red blush coloration overall, which gives it a more attractive and unique appearance in the constellation of the various Fuji varieties.

‘Swedes Fuji’ is a new, unique and strikingly different variation of the standard Fuji apple tree (Malus domestica). It is characterized by its brilliant red fruit color, with absolutely no striping pattern (seen on standard Fujis). The fruit is medium to large in size and tastes similar to, if not slightly sweeter than standard Fujis. ‘Swedes Fuji’ is of the generally late season harvest variety. It is easier to harvest though, than the standard Fuji, because throughout an entire orchard most of the fruit tends to have similar coloration and maturation rates, resulting in almost all of the fruit being more uniform in color at harvest time.

### FIGURES

### All in Color

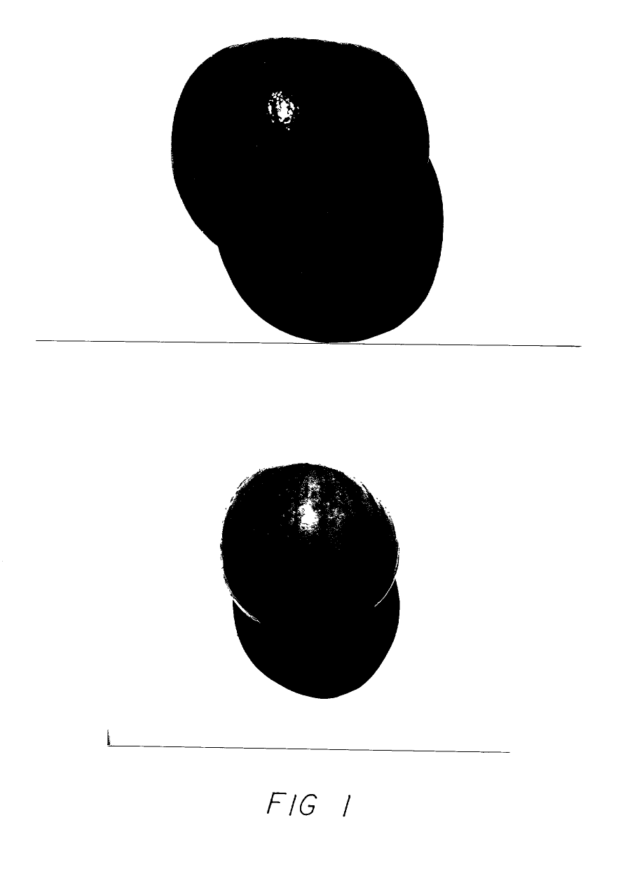

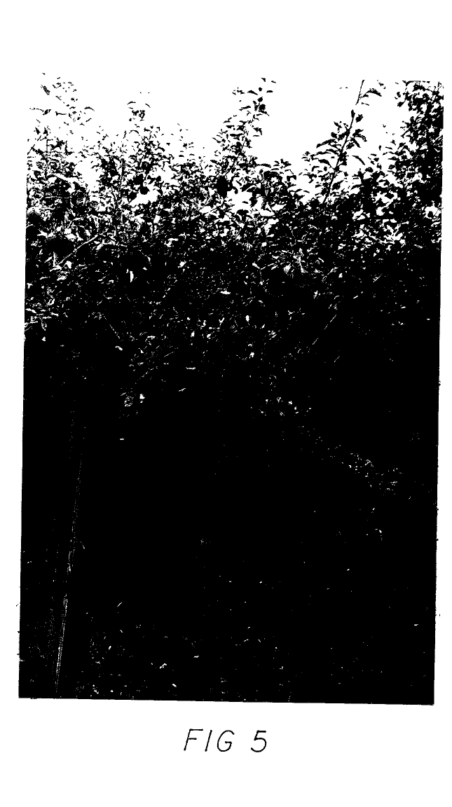

FIG. 1 shows a typical example of the ‘Swedes Fuji apple (upper). It demonstrates the uniformly brilliant color over the entire apple with no striping pattern. This coloration is found on nearly all apples throughout each entire tire. (See FIG. 5 on following pages.) For comparison purposes an example of the standard Fuji variety is shown (lower) demonstrating its well known striping pattern and subdued colors.

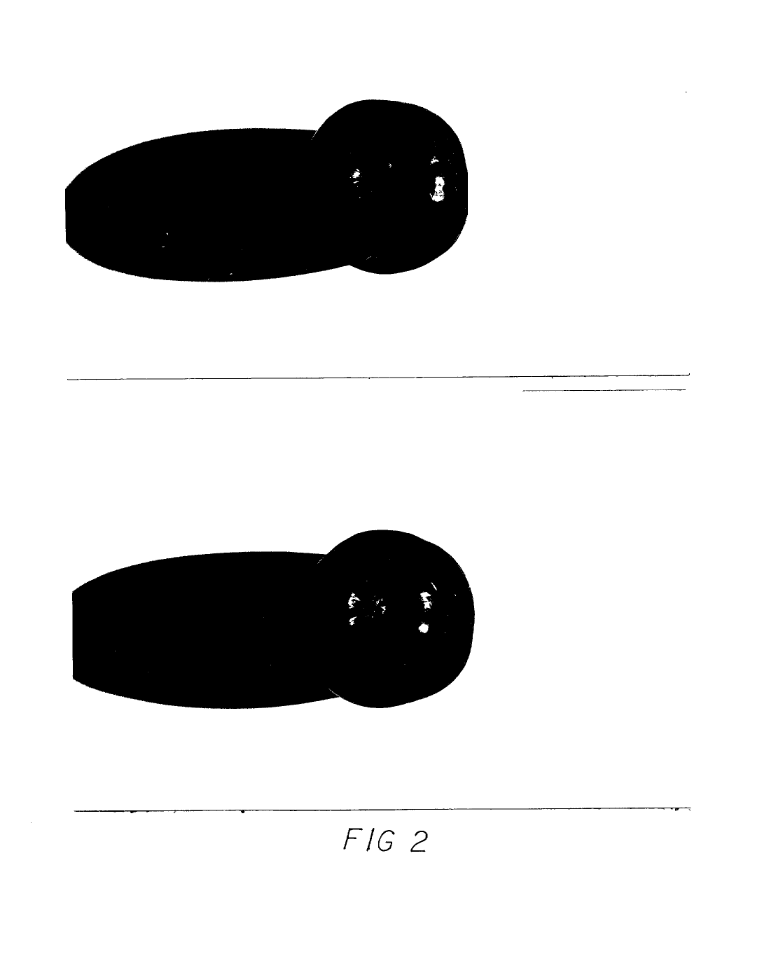

FIG. 2 shows top and bottom views of the ‘Swedes Fuji’ fruit demonstrating the extend of the blush coloration completely into the stem basin and the calyx.

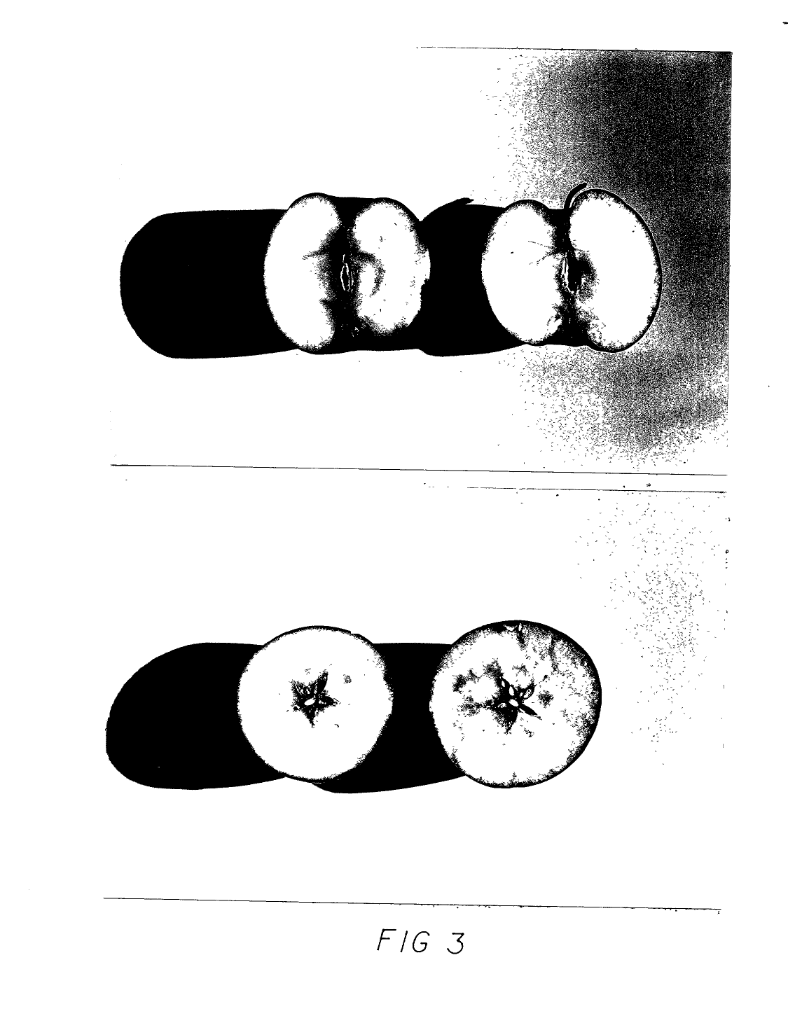

FIG. 3 shows half sections of the ‘Swedes Fuji’ cut along longitudinal (upper) and tangential (lower) axes.

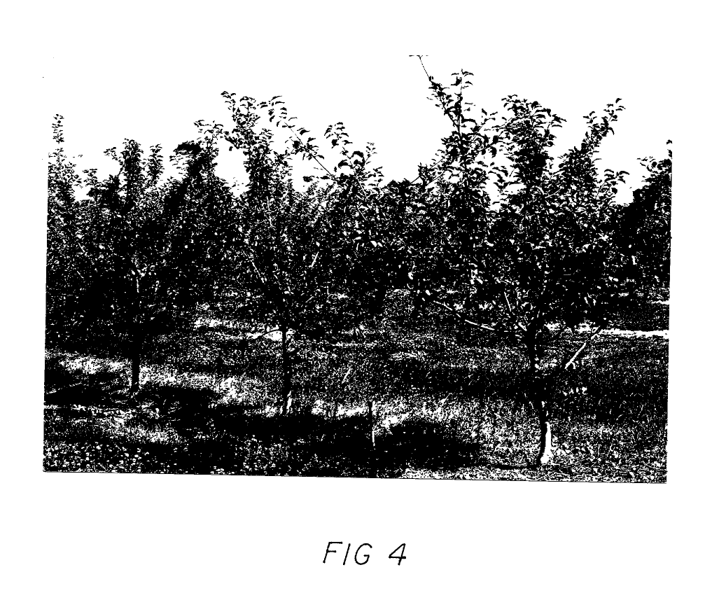

FIG. 4 shows a view of typical ‘Swedes Fuji trees in late summer.

FIG. 5 shows a typical ‘Swedes Fuji’ tree at harvest time.

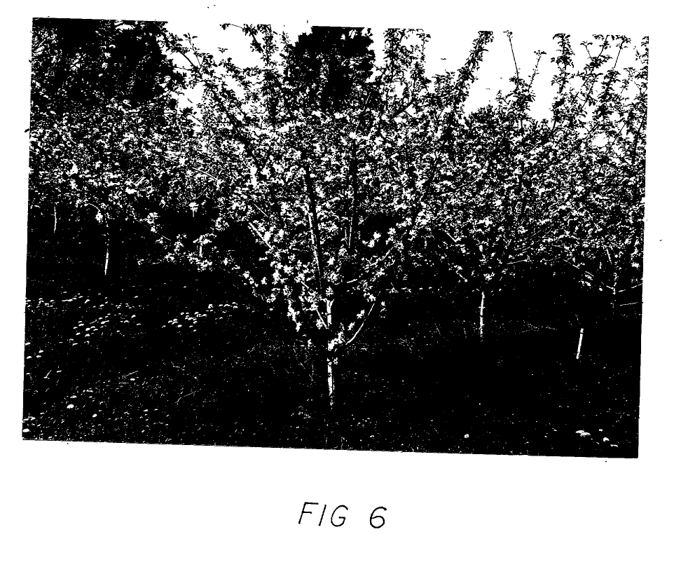

FIG. 6 shows typical ‘Swedes Fuji’ trees during full blossom.

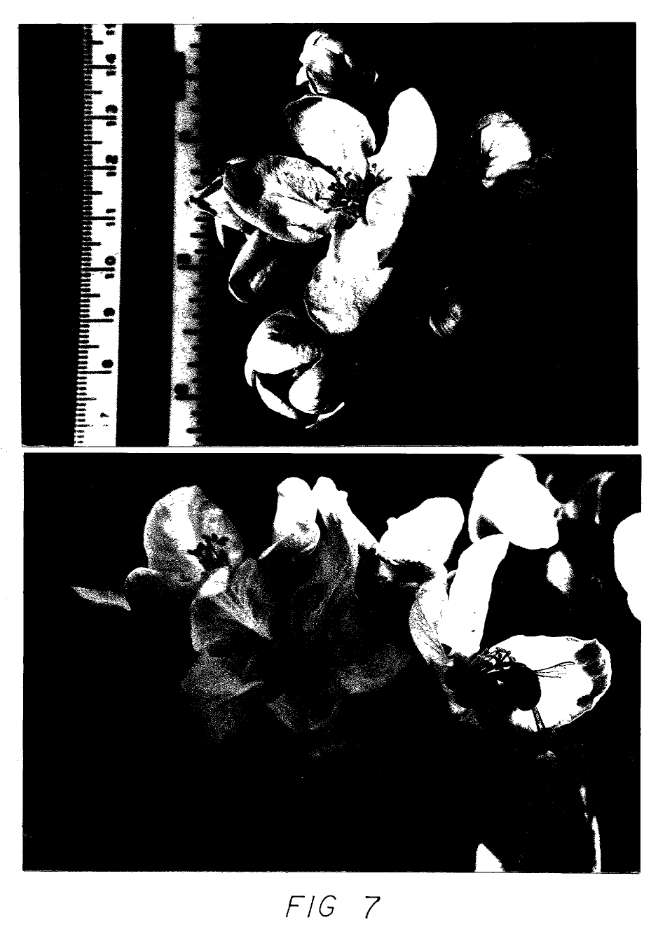

FIG. 7 shows close up views of two blossom clusters of the ‘Swedes Fuji’ apple tree.

### DETAILED DESCRIPTION

Tree:

Genus.—Malus domestica.

Designated name.—‘Swedes Fuji’.

Growing location.—Okanogan Valley in Washington state.

Soil type.—Sandy loam.

Propagation.—Asexual, retains unique characteristics through successive generations onto M7 rootstalk.

Origin/parentage.—A mutation from the traditional standard (unpatented) Fuji.

Tree Size.—Medium, with vigor similar to standard Fuji.

Productivity.—Good to excellent; needs fruit thinning in late spring (both manual and/or chemical spray applications acceptable). Tends to be somewhat more fruit on alternate years, but sometimes a good crop every year. On M7 rootstalk cultivar bears significant quantities of fruit by 3rd year.

Annular growth.—Good, needs yearly selective winter pruning.

Pollinization.—Mostly Granny Smiths were tested which performed satisfactorily; also standard Fujis performed satisfactorily, but no extensive testing done yet on these.

General appearance.—Similar to standard Fuji.

Trunk.—Medium, smooth, stocky and grey in color.

Branching pattern.—A spreading wide angle branching pattern which when accompanied with appropriate pruning tends to provide a more open canopy, allowing more sunlight to reach most all fruit on the tree. See FIGS. 4 and 5.

Current season's shoots.—Similar to standard Fuji, a weak pubescence, medium internode length, and upward extending medium length leaves.

Dormant shoots.—Similar to standard Fuji, medium length and greyish brown in color.

Leaves.—Similar to standard Fuji; for an average primary leaf, length=70 to 100 mm, width=35 to 60 mm. Configuration is rather oblong with an acuminate trip. Edges are serrated. Petioles are in general non remarkable, again similar to standard Fuji. There is medium glossiness on leaf upper surface and mild pubescence on under surface. Color is dark green on upper and pale green on lower surfaces. Stipules, paired and typically vary in length from 8 to 11 mm, with width of 1.5 to 2 mm.

Lenticals.—Medium size, non remarkable.

Growing season.—A later maturing apple with full maturity usually occurring approximately in the 3rd or 4th week of October (somewhat dependent upon weather conditions during summer growing months).

Heat/sun tolerance.—The fruit of this cultivar is less prone to sunburn than other Fuji varieties.

Growing condition requirements.—Similar as for standard Fujis.

Marketability.—An improved Fuji, perhaps mildly so in taste, but markedly so visually/esthetically. It has a surprisingly striking appearance. It processes well in the usual apple warehouses, is suitable for snacks, health foods, desserts and it could be described as a specialty premium apple with excellent quality.

Fruit:

Color appearance.—50R 4/16 in the Munsell color indication system. This is a rather brilliant red, more so than any other Fuji. (FIGS. 1 & 2).

Pattern appearance.—None. The entire surface of the apple presents a mostly uniform blush, on 95% up to 100% of the surface of the fruit. this is in contrast to the 25% to 40% usually observed on standard Fujis. The uniform coloration of the ‘Swedes Fuji’ extends completely into the stem basin and into the entire calyx. The striping pattern seen on standard Fujis is completely absent from ‘Swedes Fuji’. (FIG. 2).

Size and shape.—Medium to large, rounded smoothly curved fruit. Average height=80 to 90 mm, width=80 to 90 mm. For typical specimens this ratio remains about 1:1. Weight=280-350 grams. The fruit is relatively symetrically rounded with maximum circumference usually occurring at the equator, i.e. midway between the stem basin and the calyx. There is very little crowning on either end. The stem and blossom ends both are slightly flattened and smoothly curved approximately equally, with crown and shoulders gently rounded.

Skin.—Surface is smooth, no ridging, rather glossy, and with no tendency towards cracking. Skin thickness is relatively thin.

Taste and Flesh.—Pleasantly enhanced sweet flavor. Flesh is juicy but firm, crunchy and medium in texture. It has a mild to medium aroma. Consensus of tasters is that is tastes at least equally good (perhaps slightly better) than other Fuji varieties.

Core.—Medium in size and extent. Ellipsoid shaped in geometry when viewed from a longitudinal cut. The color is a creamy whitish hue with a yellowish tinge.

Calyx.—Morphologically similar to standard Fuji.

Stem.—Medium length and thickness.

Seeds.—Total number per apple varies in a statistically bimodal distribution pattern with peaks at either 10±1 or 15±1. Tangential cross sections of the apple show 5 symetrically located seed cells with usually 2 seeds per cell for an entire apple, or 3 seeds per cell for an entire apple. The shape of each seed is a bilaterally symetric tear drop configuration, but somewhat flattened along the long axis. A typical seed exhibits dimensions of length=8 mm, width=5 mm, and thickness=3 mm. They are a dark brown color.

Genetic composition.—Currently, patent application procedures do not require DNA testing because there exists no publically accessible DNA data bases for comparisons. Never-the-less, we were informed that our new cultivar is genetically unique, i.e. different than all other apple trees. This information came from a private company DNA test done by one of the people interested in marketing our apple. The genetic composition was evidently compared with private data bank records.

Storage life.—Keeps well with long storage life; up to 6 months in refrigeration and up to 12 months in controlled atmosphere facilities.

Harvesting.—A definite advantage over other Fuji varieties because this new cultivar's fruit matures and deepens in color at a relatively uniform rate throughout the entire tree; sufficiently so such that color selective harvesting procedures are not necessary. Upon reaching sufficient maturity the entire trees can be completely harvested. This is unlike the other varieties of Fujis. which in order to maintain good quality control, 2 or 3 selective successive pickings are (should be) done during each harvest season. See FIG. 5 for color appearance on the tree for ‘Swedes Fuji’ at harvest time.

Water coring.—Similar to standard Fuji.

Disease resistance.—No apparent differences from standard Fujis in susceptibility to the known insects and diseases, i.e. the usual control methods for aphids, mildew, codling moth, and leaf roller are applicable.

Blossoms:

Timing.—Full bloom occurs during first week in May.

Duration.—Total time from onset until petals drop is about 7 to 10 days, dependent upon the weather, especially temperature and wind conditions.

Size.—Medium to large, typical blossom diameter=50 mm. Pedal shape is an oblong rounded mix. Pedal length=24 mm, pedal width=15 mm. Each blossom is slightly cupped.

Pedal number.—5.

Stamen and pistil.—Similar to standard Fuji.

Color.—White when opened, has an external pinkish tinge when unopened.

Sepals.—Medium sized.

Fragrance.—Mild.

Overall.—Similar to parentage from standard Fuji.

1. A newly invented and distinct cultivar from the standard (not patented) Fuji apple tree as illustrated and described herein, characterized by a fruit which is a brilliant red uniform coloration, (i.e. a full blush with literally no striping pattern).
